(19) United States
(12) Patent Application Publication
(10) Pub. No.: US 2006/0088853 A1
(43) Pub. Date: Apr. 27, 2006

(54) System and method for determining sizes of polynucleotides

(76) Inventors: Zhaowei Liu, State College, PA (US); Yiqiong Wu, Fayetteville, AR (US)

Correspondence Address: FISH & RICHARDSON PC, P.O. BOX 1022, MINNEAPOLIS, MN, 55440-1022, US

(21) Appl. No.: 11/185,916

(22) Filed: Jul. 20, 2005

Related U.S. Application Data
(63) Continuation of application No. PCT/US04/01791, filed on Jan. 23, 2004.
(60) Provisional application No. 60/441,728, filed on Jan. 23, 2003.

(51) Int. Cl. C12Q 1/68

(52) U.S. Cl. 435/6

(57) ABSTRACT

The present invention relates to a method of determining whether a length of a first polynucleotide is equal to (a) a length of a second polynucleotide or (b) a length of a third polynucleotide. A sample including the first polynucleotide is provided. A first portion of the sample is combined with an amount of the second polynucleotide to form a first mixture. A second portion of the sample is combined with an amount of the third polynucleotide to form a second, different mixture. The second polynucleotide includes at least 1 additional base than the third polynucleotide. The at least 1 additional base is located intermediate terminal ends of the second polynucleotide. First duplexes including the first polynucleotide and the second polynucleotide are prepared. Second duplexes including the first polynucleotide and the third polynucleotide are prepared. The first and second duplexes are subjected to temperature gradient electrophoresis to obtain electrophoresis data. The electrophoresis data is analyzed to determine whether the length of the first polynucleotide is equal to (a) the length of the second polynucleotide or (b) the length of the third polynucleotide.

### RELATED APPLICATIONS

The present application claims priority to U.S. Provisional application No. 60/441,728 filed Jan. 23, 2003, which provisional application is incorporated herein in its entirety.

### FIELD OF THE INVENTION

The present invention relates to the determination of the length of a polynucleotide.

### BACKGROUND

Sizing for polynucleotides to detect length differences typically relies on direct measurement by comparing migration time of a testing sample to molecular ladders either run on the same separation matrix under same conditions. This strategy requires strict running conditions and calibration, and is difficult to achieve a precise estimate when testing molecules only having a few base-pair differences.

High-throughput detection of DNA length polymorphism by capillary electrophoresis is usually performed by direct size estimation. Dye-tagged DNA fragments are mixed and co-migrated with molecular ladders of known sizes. The ladders are labeled with a different dye so that the fluorescence of the testing DNA fragment and the ladders can be separated into two different color channels. The size estimation of the testing fragment is through the comparison of migration time of ladders co-injected and separated in a same capillary.

However, tagging DNA fragments with fluorescent dyes is expensive. Size estimation by untagged DNA fragments and ladders separated in different capillaries is possible but unreliable due to migration variation among different capillaries. Even size estimation by co-migration along the same separation lane may generate variation since the composition of the testing DNA fragment may be different from that of the ladders. So, higher sizing resolution within 1 base pair would require second calibration ladders that contain the same or highly similar sequence composition to the sample molecule.

### SUMMARY OF THE INVENTION

One embodiment of the present invention relates to a temperature gradient electrophoresis method for determining the length of a polynucleotide. The method comprises, providing a sample comprising a first polynucleotide, which may be, for example, a PCR product. The first polynucleotide may be single stranded or double stranded. A first portion of the of the sample and a second polynucleotide are combined to prepare a first mixture. A second portion of the sample and a third polynucleotide are combined to prepare a second mixture. The second polynucleotide comprises at least 1 additional base than the third polynucleotide. The at least 1 additional base is located intermediate terminal ends of the second polynucleotide.

First duplexes comprising the first polynucleotide and the second polynucleotide are prepared. Second duplexes comprising the first polynucleotide and the third polynucleotide are prepared. The first and second duplexes are subjected to temperature gradient electrophoresis to obtain electrophoresis data. The electrophoresis data is analyzed to determine whether the length of the first polynucleotide is equal to (a) the length of the second polynucleotide or (b) the length of the third polynucleotide.

In any embodiment in accordance with the invention, the temperature gradient electrophoresis may be temperature gradient capillary electrophoresis. The first and second duplexes may be subjected to temperature gradient electrophoresis in the same or in different capillaries. The polynucleotides may be subjected to temperature gradient electrophoresis in the presence of an intercalating dye.

The second and third polynucleotides may each comprise more than 500 bases, more than 750 bases, more than 1000 bases, or more than 1250 bases. The second polynucleotide may comprise at least 2 additional bases than the third polynucleotide. The at least 2 additional bases are preferably located intermediate terminal ends of the second polynucleotide. The at least 2 additional bases may be consecutive. The second polynucleotide, intermediate its terminal ends, may comprise less than 20 additional bases than the third polynucleotide, less than 15 additional bases, less than 10 additional bases, or less than 5 additional bases.

The electrophoresis data may comprise a number N1 first peaks indicative of the presence of the first duplexes, N1 being 1 or greater, and a number N2 second peaks indicative of the presence of the second duplexes, N2 being 1 or greater. The step of analyzing the electrophoresis data may comprise determining N1and N2. If N1>N2, the length of the first polynucleotide is preferably determined to be equal to the length of the third polynucleotide. If N1<N2, the length of the first polynucleotide is preferably determined to be equal to the length of the second polynucleotide.

The step of analyzing the electrophoresis data may comprise determining a total width of the N1 first peaks and a total width of the N2 second peaks. The total width may be determined, for example, on the basis of a portion of the maximum intensity of the peaks. If the total width of the N1 first peaks > the total width of the N2 second peaks, the length of the first polynucleotide is preferably determined to be equal to the length of the third polynucleotide. If the total width of the N1 first peaks < the total width of the N2 second peaks, the length of the first polynucleotide is preferably determined to be equal to the length of the second polynucleotide.

The step of analyzing the electrophoresis data may comprise determining a migration rate of the N1 first peaks and a migration rate of the N2 second peaks. If the migration rate of the N1 first peaks is > the migration rate of the N2 second peaks, the length of the first polynucleotide is preferably determined to be equal to the length of the third polynucleotide. If the migration rate of the N1 first peaks is < the migration rate of the N2 second peaks, the length of the first polynucleotide is preferably determined to be equal to the length of the second polynucleotide.

The step of analyzing the electrophoresis data may comprise determining a migration time of the N1 first peaks and a migration time of the N2 second peaks. If the migration time of the N1 first peaks is < the migration time of the N2 second peaks, the length of the first polynucleotide is preferably determined to be equal to the length of the third polynucleotide. If the migration time of the N1 first peaks is > the migration time of the N2 second peaks, the length of the first polynucleotide is preferably determined to be equal to the length of the second polynucleotide.

The first, second, and third polynucleotides may be of substantially the same allele.

Another embodiment of the invention relates to a method of determining a size of a first polynucleotide. The method comprises subjecting a plurality of first duplexes to temperature gradient electrophoresis to obtain first electrophoresis data comprising a number N1 first peaks indicative of the presence of the first duplexes, N1 being 1 or greater. Each of the first duplexes preferably comprises the first polynucleotide and a second polynucleotide. A plurality of second duplexes are subjected to temperature gradient electrophoresis to obtain second electrophoresis data comprising a number N2 second peaks indicative of the presence of the second duplexes, N2 being 1 or greater. The second duplexes preferably comprise the first polynucleotide and a third polynucleotide. The second polynucleotide comprises at least 1 additional base than the third polynucleotide. The at least 1 additional base is located intermediate terminal ends of the second polynucleotide.

The N1 first peaks and the N2 second peaks are compared to determine whether a length of the first polynucleotide is equal to the length of the second polynucleotide or to the length of the third polynucleotide.

The step of comparing may comprise determining N1 and N2. If N1>N2, the length of the first polynucleotide is preferably determined to be equal to the length of the third polynucleotide. If N1<N2, the length of the first polynucleotide is preferably determined to be equal to the length of the second polynucleotide.

The step of comparing may comprise determining a total width of the N1 first peaks and a total width of the N2 second peaks. If the total width of the N1 first peaks > the total width of the N2 second peaks, the length of the first polynucleotide is preferably determined to be equal to the length of the third polynucleotide. If the total width of the N1 first peaks < the total width of the N2 second peaks, the length of the first polynucleotide is preferably determined to be equal to the length of the second polynucleotide.

The step of comparing may comprise determining a migration rate of the N1 first peaks and a migration rate of the N2 second peaks. If the migration rate of the N1 first peaks is < the migration rate of the N2 second peaks, the length of the first polynucleotide is preferably determined to be equal to the length of the third polynucleotide. If the migration rate of the N1 first peaks is > the migration rate of the N2 second peaks, the length of the first polynucleotide is preferably determined to be equal to the length of the second polynucleotide.

The step of comparing may comprise determining a migration time of the N1 first peaks and a migration time of the N2 second peaks, wherein (a) if the migration time of the N1 first peaks is > the migration time of the N2 second peaks, the length of the first polynucleotide is determined to be equal to the length of the third polynucleotide and (b) if the migration time of the N1 first peaks is < the migration time of the N2 second peaks, the length of the first polynucleotide is determined to be equal to the length of the second polynucleotide.

The second and third polynucleotides may each comprise more than 500 bases, more than 750 bases, or more than 1000 bases. The second polynucleotide may comprise at least 2 additional bases than the third polynucleotide. The at least 2 additional bases are preferably located intermediate terminal ends of the second polynucleotide. The at least 2 additional bases may be consecutive. The second polynucleotide, intermediate its terminal ends, may comprise less than 20 additional bases than the third polynucleotide, less than 15 additional bases, less than 10 additional bases, or less than 5 additional bases.

Another embodiment of the invention relates to a computer-readable medium comprising executable software code, the code for determining whether a length of a first polynucleotide is equal to (a) a length of a second polynucleotide or (b) a length of a third polynucleotide. The computer readable medium comprises: code to receive first electrophoresis data, the first electrophoresis data having been obtained by subjecting first duplexes to temperature gradient electrophoresis, the first duplexes comprising the first polynucleotide and, at least partially paired therewith, the second polynucleotide; code to receive second electrophoresis data, the second electrophoresis data having been obtained by subjecting second duplexes to temperature gradient electrophoresis, the second duplexes comprising the first polynucleotide and, at least partially paired therewith, the third polynucleotide, the second polynucleotide comprising at least 1 additional base than the third polynucleotide, the at least 1 additional base being located intermediate terminal ends of the second polynucleotide; code to determine the presence of a number N1 first peaks in the first electrophoresis data, the N1 first peaks being indicative of the presence of the first duplexes, N1 being 1 or greater;

code to determine the presence of a number N2 second peaks in the second electrophoresis data, the N2 second peaks being indicative of the presence of the second duplexes, N2 being 1 or greater; and code to determine whether the length of the first polynucleotide is equal to (a) the length of the second polynucleotide or (b) the length of the third polynucleotide based upon the N1 first peaks and the N2 second peaks.

The code to determine whether the length of the first polynucleotide is equal to (a) the length of the second polynucleotide or (b) the length of the third polynucleotide may comprise code to determine the number N1 and the number N2; code to determine that the length of the first polynucleotide is equal to the length of the third polynucleotide if N1>N2; and code to determine that the length of the first polynucleotide is equal to the length of the second polynucleotide if N1<N2.

The code to determine whether the length of the first polynucleotide is equal to (a) the length of the second polynucleotide or (b) the length of the third polynucleotide may comprise: code to determine a total width of the N1 first peaks and a total width of the N2 second peaks; code to determine that the length of the first polynucleotide is equal to the length of the third polynucleotide if the total width of the N1 first peaks > the total width of the N2 second peaks; and code to determine that the length of the first polynucleotide is equal to the length of the second polynucleotide if the total width of the N1 first peaks < the total width of the N2 second peaks.

The code to determine whether the length of the first polynucleotide is equal to (a) the length of the second polynucleotide or (b) the length of the third polynucleotide may comprise: code to determine a migration rate of the N1 first peaks and a migration rate of the N2 second peaks; code to determine that the length of the first polynucleotide is equal to the length of the second polynucleotide if the migration rate of the N1 first peaks > the migration rate of the N2 second peaks; and code to determine that the length of the first polynucleotide is equal to the length of the third polynucleotide if the migration rate of the N1 first peaks < the migration rate of the N2 second peaks.

The code to determine whether the length of the first polynucleotide is equal to (a) the length of the second polynucleotide or (b) the length of the third polynucleotide may comprise: code to determine a migration velocity of the N1 first peaks and a migration velocity of the N2 second peaks; code to determine that the length of the first polynucleotide is equal to the length of the second polynucleotide if the migration velocity of the N1 first peaks < the migration velocity of the N2 second peaks; and code to determine that the length of the first polynucleotide is equal to the length of the third polynucleotide if the migration velocity of the N1 first peaks > the migration velocity of the N2 second peaks.

Another embodiment of the invention relates to a method of determining a size of a first polynucleotide. The method comprises receiving first electrophoresis data, the first electrophoresis data having been obtained by subjecting a plurality of first duplexes to temperature gradient electrophoresis, the first electrophoresis data comprising a number N1 first peaks indicative of the presence of the first duplexes, N1 being 1 or greater, wherein each of the first duplexes comprise the first polynucleotide and a second polynucleotide. Second electrophoresis data is received. The second electrophoresis data having been obtained by subjecting a plurality of second duplexes to temperature gradient electrophoresis, the second electrophoresis data comprising a number N2 second peaks indicative of the presence of the second duplexes, N2 being 1 or greater, wherein the second duplexes comprise the first polynucleotide and a third polynucleotide, the second polynucleotide comprising at least 1 additional base than the third polynucleotide, the at least 1 additional base being disposed intermediate terminal ends of the second polynucleotide. The N1 first peaks and the N2 second peaks are compared to determine whether a length of the first polynucleotide is equal to the length of the second polynucleotide or to the length of the third polynucleotide.

Another embodiment of the present invention relates to a method for determining whether a sample of DNA is of a first or second genotype, the DNA comprising a first polynucleotide having a length L1 and a second polynucleotide having a length L2, wherein (a) if the DNA is of the first genotype, L1 and L2 are the same and the first and second polynucleotides are sufficiently complementary to form a first duplex and (b) if the DNA is of the second genotype, the second polynucleotide comprises at least 1 additional base than the first polynucleotide, the at least 1 additional base being located intermediate terminal ends of the second polynucleotide so that a second duplex comprising the first and second polynucleotides comprises an unpaired region associated with the at least 1 additional base, the unpaired region being located intermediate terminal ends of the second duplex. The method comprises combining the first and second polynucleotides with a first control sample comprising a third polynucleotide and a complementary fourth polynucleotide to prepare a first mixture. Both the third and the fourth polynucleotides preferably have the same length, either L1 or L2, so that a duplex comprising the third and fourth polynucleotides lacks an unpaired region located intermediate terminal ends of the duplex.

Combined polynucleotides of the first mixture are subjected to at least one melting step and one annealing step to prepare a second mixture comprising (a) a duplex comprising the first polynucleotide and one of the third and fourth polynucleotides and (b) a duplex comprising the second polynucleotide and the other of the third and fourth polynucleotides.

Duplexes of the second mixture are subjected to temperature gradient electrophoresis to obtain first electrophoresis data. Whether the DNA is of the first or second genotype is determined based on the first electrophoresis data.

The method may further comprise combining the first and second polynucleotides with a second control sample comprising a fifth polynucleotide and a complementary sixth polynucleotide to prepare a third mixture, both the fifth and the sixth polynucleotides have the same length L1 if the third and fourth polynucleotides have length L2 or the same length L2 if the third and fourth polynucleotides have length L1, so that a duplex comprising the fifth and sixth polynucleotides lacks an unpaired region located intermediate terminal ends of the duplex.

Combined polynucleotides of the third mixture are subjected to at least one melting step and one annealing step to prepare a fourth mixture comprising (a) a duplex comprising the first polynucleotide and one of the fifth and sixth polynucleotides and (b) a duplex comprising the second polynucleotide and the other of the fifth and sixth polynucleotides.

Duplexes of the fourth mixture are subjected to temperature gradient electrophoresis to obtain second electrophoresis data. Whether the DNA is of the first or second genotype is determined based on both the first and the second electrophoresis data.

The first electrophoresis data preferably comprises a number N1 peaks indicative of the presence of the duplexes of the second mixture, N1 being 1 or greater. The N1 peaks of the first electrophoresis data preferably have respective first intensities. The second electrophoresis data preferably comprises a number N2 peaks indicative of the presence of the duplexes of the fourth mixture, N2 being 1 or greater. The N2 peaks of the second electrophoresis data preferably have respective second intensities. The method preferably comprises determining N1 and N2 and, optionally, determining the respective first and second intensities. Whether the DNA is of the first or second genotype is determined based upon at least 1 of N1, N2, the respective first intensities, and the respective second intensities.

The method may comprise subjecting the DNA, in the absence of the third and fourth polynucleotides, to at least one melting step and at least one annealing step. Subjecting the melted annealed DNA to temperature gradient electrophoresis to obtain sample electrophoresis data and determining whether the DNA is of the first or second genotype based on both the first and third electrophoresis data.

### BRIEF DESCRIPTION OF THE DRAWINGS

The present invention is discussed below in reference to the Drawings in which:

FIG. 1 shows duplexes prepared from a sample polynucleotide and a control polynucleotide having different lengths;

FIG. 2 shows electrophoresis data obtained duplexes prepared from a sample polynucleotide and a control polynucleotide having different lengths and electrophoresis data obtained duplexes prepared from a sample polynucleotide and a control polynucleotide having the same lengths;

FIG. 3 shows a device for performing temperature gradient electrophoresis in accordance with the present invention;

FIGS. 4 and 5 show a plurality of electrophoresis data obtained in accordance with the present invention;

FIG. 6 shows, schematically, a method for determining a size of a polynucleotide in accordance with the present invention; and

FIGS. 7a-7c and 8a-8c show, schematically, a method for determining a genotype of a polynucleotide in accordance with the present invention.

### DETAILED DESCRIPTION OF THE PREFERRED EMBODIMENT

DNA markers with a deletion/insertion (indel) or a different number of simple sequence repeat units (e.g., a microsatellite) are widely used in genetic analysis, in disease diagnosis, and in environmental monitoring of various organisms.

These markers are usually amplified with a pair of primers (20-50 bases of oligonuleotides) for the individuals in a population of interest. Amplified DNA products often represent different alleles for a locus. Thus, the comparing DNA fragments are usually identical in DNA sequence, except in the region of indel, which is located inside of two priming sites, not at two sequence ends. Therefore, the mismatched site occurs intermediate the ends of the duplex and intermediate the ends of the individual polynucleotides of the duplex. The present invention applies temperature gradient electorphoresis to determine the presence of such length differences.

The present invention relates to a method for determining the size of a sample polynucleotide by heteroduplex analysis with temperature gradient electrophoresis (TGE). The sample polynucleotide may be a single polynucleotide or a double stranded polynucleotide (a duplex). In a preferred method, a polynucleotide with a known size serves as a control, which may be a single stranded polynucleotide or a duplex. Both the control and sample polynucleotides may be products of a polymerase chain reaction amplification. The control polynucleotide has a sequence complementary to that of the sample polynucleotide. The sample and control polynucleotides are combined and subjected to at least one denaturation and annealing step to prepare duplexes comprising the sample and control polynucleotides.

If the sample and control polynucleotides are single stranded, a single duplex is formed. If the sample and control polynucleotides are double stranded, four duplexes, which may or may not be different, are formed. If the sample and control polynucleotides includes polynucleotides of different length, a duplex comprising the different length polynucleotides may exhibit an unpaired region. The unpaired region reduces the stability and, therefore, the melting point of the duplex. The melting point is the temperature at which half of the strands of a sample of double stranded polynucleotide melt. In accordance with the present invention, TGE exploits the difference in stability to determine whether polynucleotides of the sample have the same or different length as polynucleotides of the control. If the length of the control polynucleotide is known, the length of the sample polynucleotide can be determined from the TGE.

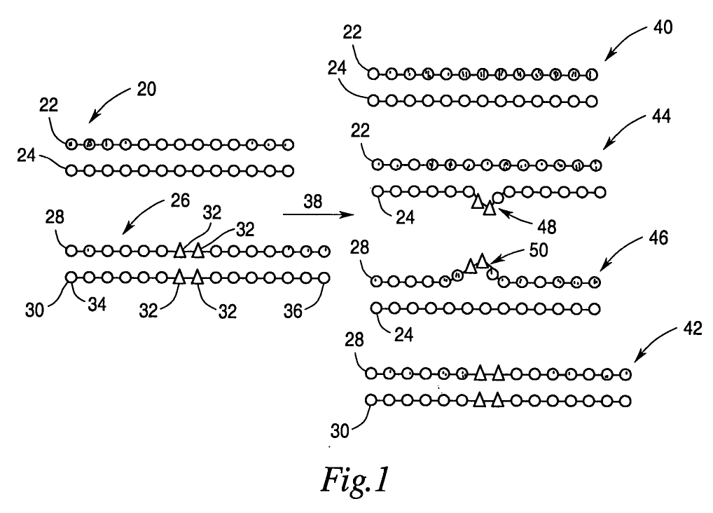

Referring to FIG. 1, duplexes formed from a sample polynucleotide and a control polynucleotide have an unpaired region. A sample polynucleotide 20 is a duplex comprising first 22 and second 24 polynucleotide strands each having a length of 13 bases. A control polynucleotide 26 is a duplex comprising first 28 and second 30 control polynucleotide strands each having a length of 15 bases. Each of the first and second control polynucleotide strands include two additional bases 32 located intermediate terminal ends 34, 36 of control polynucleotide 26. Upon subjecting the sample polynucleotide 20 and control polynucleotide 26 to at least one melting and annealing step 38, the polynucleotide strands 22, 24, 28, and 30 combine to form 4 duplexes. A first duplex 40 is identical to sample polynucleotide 20. A second duplex 42 is identical to control polynucleotide 26. A third duplex 44 comprises a polynucleotide strand 22 and a polynucleotide strand 30. A fourth duplex 46 comprises a polynucleotide strand 28 and a polynucleotide strand 24. Third duplex 44 comprises an unpaired region 48. Fourth duplex 46 comprises an unpaired region 50. In contrast to the situation in FIG. 1 in which the sample and control polynucleotides have different lengths, sample and control polynucleotides of the same length will form essential identical duplexes without unpaired regions.

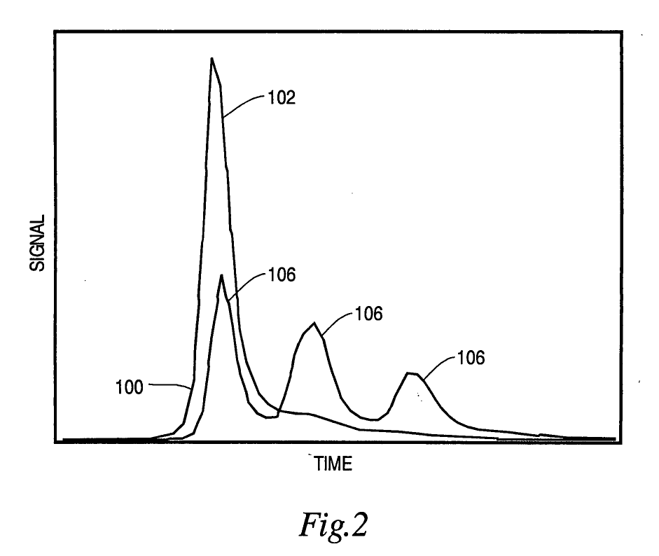

Referring to FIG. 2, and as discussed above, TGE, in accordance with the present invention, exploits the presence or absence of unpaired region to allow the size of a sample polynucleotide to be determined. If the sample has an identical length to that of the control, electrophoresis data from TGE will include a single-peak indicative of the presence of the duplexes. Electrophoresis data 100 includes a single peak 102 resulting from the TGE of duplexes prepared from a sample polynucleotide having a length of 290 base pairs and a control polynucleotide having a length of 290 base pairs. If the sample does not have an identical length to that of the control, electrophoresis data from TGE may include multiple peaks, a broader set of peaks, and/or a reduced migration time compared to electrophoresis data from the identical length situation. Electrophoresis data 104 includes 3 peaks 106 resulting from the TGE of duplexes prepared from the sample polynucleotide having a length of 290 base pairs and a control polynucleotide having a length of 292 base pairs. As can be seen, electrophoresis data 104 includes more peaks that are spread across a greater migration time than the peak 102 of electrophoresis data 100. Thus, in accordance with the present invention, size information is not obtained through estimation. Instead, size determination may be made on the basis of simple assignment based on peaks. Thus, the present invention provides a low cost method of genetic analysis of any organism and, for example, in disease diagnosis linked to insertion/deletion differences between DNA markers.

The melting point of a duplex of a pair of polynucleotides depends at least in part on the presence or absence of an unpaired region in the duplex, the length of the duplex, and the position along the duplex of the unpaired region, if present. The thermal stability of the duplex and, therefore, the melting temperature, depends at least in part on the degree of complementarity between the polynucleotides of the duplex. For example, the presence of even one additional base (such as may be caused by an insertion) along one polynucleotide of a duplex as compared to the other polynucleotide of the duplex is sufficient to reduce the melting temperature of the complex compared to the fully matched duplex.

In accordance with the present invention, temperature gradient electrophoresis (TGE), e.g., temperature gradient capillary electrophoresis (TGCE), exploits differences in the melting temperatures of a duplex comprising an unpaired region as compared to an otherwise similar duplex lacking the unpaired region. During TGE, the temperature is increased from a temperature below the melting point of all duplexes present in the mixture to a temperature preferably, but not necessarily, greater than the melting point of all duplexes present. As the temperature is increased, heteroduplexes comprising an unpaired region exhibit disruption of secondary structure and melting prior to homoduplexes lacking the unpaired region because the base unpaired region reduces the strength of binding between the polynucleotides of the heteroduplex. As the secondary structure becomes disrupted and the a duplex begins to melt, its electrophoretic mobility is retarded. Because this occurs at a lower temperature for heteroduplexes than homoduplexes, the two types of duplexes can be separated from one another. The separated duplexes may be detected, such as by using laser-induced fluorescence or other optical detection method. The detection provides electrophoresis data, which may contain peaks indicative of the presence of the duplexes.

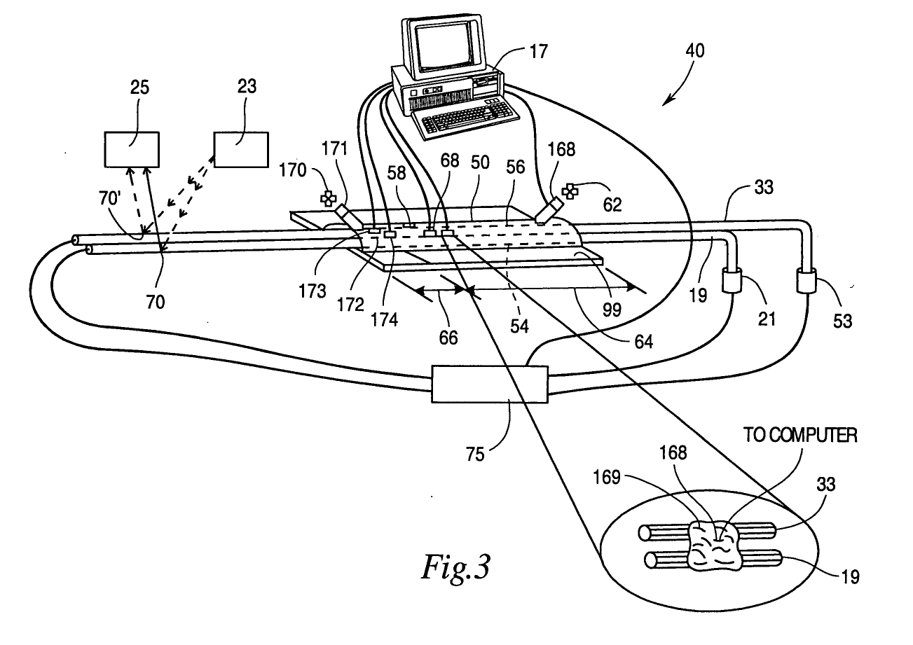

Referring to FIG. 3, a preferred arrangement of an embodiment of a temperature gradient electrophoresis device 40 for determining a size of polynucleotides is shown. A sample capillary 33 is provided to electrophoretically separate duplexes. By capillary it is meant any structure configured and arranged to separate a sample using electrophoresis. Preferred structures include capillaries, microfabricated channels, and planar structures, such as lanes of slab gels. In one embodiment, the present invention excludes the use of slab gels and other planar structures lacking well-defined, distinct separation lanes.

Capillary 33 is arranged to be in fluid contact with a sample reservoir 53, which is configured to contain a volume of sample sufficient to perform an analysis. Examples of suitable sample reservoirs include the wells of a microtitre plate, a structure configured to perform PCR amplification on a volume of sample, a reservoir of a microfabricated lab on a chip device, and the like.

Device 40 is provided with a power supply 75 suitable for providing a sufficient voltage and current for electrophoretic separation of a sample. The power supply is preferably configured to allow at least one of the current or resistance of the capillary to be monitored during a separation. Preferably, the current or resistance data is received by the computing device 17 to allow the electric potential to be varied to maintain a constant current or resistance. This is discussed in more detail below.

A temperature controlled portion 54 of sample capillary 33 is arranged to be in thermal contact with a heat source such as a hot plate 99, or the like. Temperature controlled portion 54 has a length 64. Optionally, or in addition, the external heat source may comprise a wire, filament, or other ohmic heating element arranged external to the capillary. A temperature sensitive device such as a thermocouple 168 is disposed in thermal contact with capillary 33 and reference capillary 19 to determine the temperature of migrating species therein. Thermocouple 168 is in communication with computing device 17, which can adjust the temperature of hotplate 99 to maintain or establish a predetermined temperature or temperature profile.

Alternatively or in combination with hotplate 99, mutation detection system 40 may include an element 62 to cause temperature controlled gas or liquid to flow in thermal contact with capillary 33. The gas or liquid enters at an input port 268 and exits at an exit port 58. The capillary is preferably surrounded by a thermally conductive medium, such as a thermally conductive paste 169, to enhance thermal contact between the heating element and the capillary. Capillary 33 may have a cooled portion 172 having a length 66 to reduce the temperature of migrating compounds prior to detection. An element 170 may be provided to introduce chilled gas or liquid to cooled portion 172 through an entry port 171.

Device 40 is preferably provided with an optional reference capillary 19 configured to simultaneously separate a reference sample comprising reference polynucleotides. Reference capillary 19 includes a reference reservoir 21 configured to contain the reference sample. Sample and reference capillaries 33 and 19 include respective detection zones 70′ and 70.

Device 40 also includes a light source 23, such as a laser emitting a wavelength suitable to generate a spectroscopic signal, such as fluorescence or absorbance from separated duplexes. A detector 25 is arranged to detect the spectroscopic signal, which is converted to electrophoresis data representative of the spectroscopic signal. The electrophoresis data are sent to computing device 17. The electrophoresis data can be represented by, for example, a time-spectroscopic intensity plot including peaks indicative of the presence of a duplex. A specific example is an electropherogram including a time-fluorescence intensity plot. The fluorescence may result from an intercalating dye intercalated with the duplex. In this embodiment, it is preferred that the temperature of the detection zone be less than the melting temperature of duplexes to be detected. Another example of electrophoresis data is an electropherogram including a time-absorbance intensity plot where the absorbance relates to an attenuation of light by the duplexes. For a time-absorbance measurement, a detector is disposed to measure the intensity of light that has passed generally radially through the separation lane.

The TGE is preferably conducted using an electrophoresis medium, such as a sieving medium, that separates migrating species on the basis of size and/or shape. An example of a suitable sieving medium is an electrophoresis gel. The electrophoresis is preferably carried out within the bore of a capillary. Within the bore, materials migrate substantially along a migration axis under the influence of an electric field.

A preferred separation medium for mutation detection comprises a buffer, such as TBE buffer, which can be prepared, for example, by dissolving 8.5 g premixed TBE buffer powder (Amerosco, Solon, Ohio.) into 500 ml dionized water.

An electrophoresis medium, such as a sieving matrix, can be prepared using polyvinylpyrrolidone (PVP) which is available from Sigma (St. Louis, Mo.). A preferred sieving matrix can be made by dissolving about 0.5% to about 6% (w/v) of 360,000 M PVP into TBE buffer. Preferably, the amount of PVP is about 3% (w/v). The viscosity of a three percent solution is less than 10 cp. Alternatively the separation medium includes other sieving matrices such as polyacrylamide gels.

In one embodiment, the electrophoretic separation medium comprises an intercalating dye, such as ethidium bromide to allow fluorescence detection of the separated polynucleotides. The intercalating dye preferentially allows detection of double stranded DNA (e.g., duplexes) as compared to single stranded DNA. In one embodiment, the separation medium and polynucleotides are substantially free of a covalent tag suitable for fluorescence detection of single strands of DNA. By substantially free it is meant that the presence of any covalent tag suitable for fluorescence detection of single strands of DNA is insufficient to interfere with the detection of sample compounds using fluorescence resulting from the intercalating dye. In one embodiment, the polynucleotides to be separated are preferably substantially free of fluorescent dyes that covalently tag single stranded DNA. Multiple samples comprising polynucleotides, such as DNA fragments, can be simultaneously analyzed.

During temperature gradient electrophoresis, there is preferably at least one change in the temperature of the separation medium as a function of time. Temperatures can be varied over any time and temperature range sufficient to induce a mobility differential between duplexes to be separated. Preferred temperature extremes include a minimum of at least about 0° C. and a maximum of about 100° C. Preferably, the temperature within the temperature control zone is substantially constant along a dimension of the separation medium that is perpendicular to the direction of migration. By substantially constant temperature it is meant that the spatial temperature variations are insufficient to introduce measurable mobility variations for compounds disposed at different spatial locations within the temperature control zone at any given instant. Thus, at any given instant, the temperature at any point along the portion of each capillary within the temperature control zone is preferably constant, i.e., there are substantially no spatial temperature gradients in the temperature control zone.

A duplex containing an unpaired region intermediate terminal ends of the duplex (defined herein as a heteroduplex) will exhibit disruption of secondary structure and melting at a lower temperature than a duplex lacking such an unpaired region (defined herein as a homoduplex). Therefore, in an sieving electrophoresis medium, such as a gel or a long chain linear polymer solution, the heteroduplex and homoduplex complexes can be separated or otherwise distinguished by providing, for at least a portion of the electrophoresis, a temperature sufficient to disrupt and/or melt the heteroduplex complex but not the homoduplex complex.

Increasing the temperature of the separation medium from an initial value that is less than the melting temperature of both the homoduplex complex and the heteroduplex complex, will cause the heteroduplex complex to exhibit a retarded migration behavior near its melting temperature compared to the homoduplex complex. As the temperature is raised above the melting temperature of the homoduplex complex, the difference in mobilities between the pair of compounds is reduced. Thus, separation between a homoduplex complex and heteroduplex complex depends in part on the total amount of time the separation medium is at a temperature above the melting point of the heteroduplex complex but less than the melting temperature of the homoduplex complex. The presence or absence of an unpaired region can be identified by the difference in the resulting electrophoretic patterns between the homoduplex and the heteroduplex.

For accurate comparison of the patterns, a reproducible temperature profile is required. Because in this invention the temperature of the separation medium can be varied independently of the electric field, arbitrary temperature variation profiles can be selected. For the separation of heteroduplex complexes using an apparatus and temperature profile of the present invention, migration times have a relative standard deviation of less than 2%.

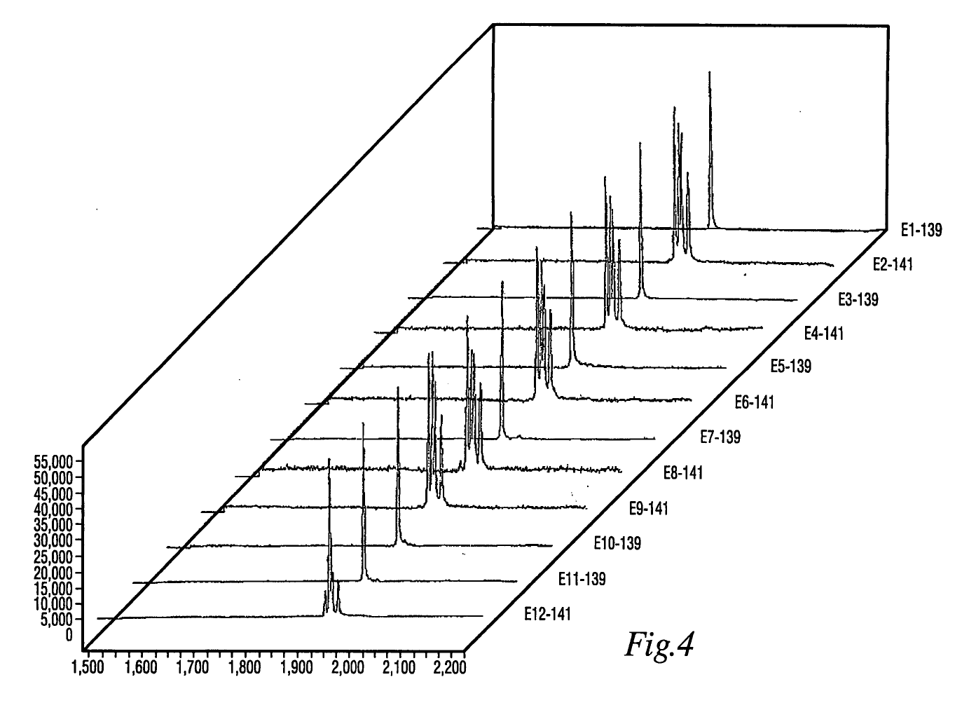

The present invention is suitable for high-throughput determination of polynucleotide size, by multiplexing large numbers of samples. Preferably, at least as many as 96 electrophoretic separations can be simultaneously performed. For example, referring to FIG. 4, 12 sets of simultaneously obtained electrophoresis data, E1-E12 are shown. Electrophoresis data E1, E3, E5, E7, E10, and E11 were obtained from the TGCE of duplexes prepared by melting an annealing a double stranded sample polynucleotide having a length of 139 base pairs in the presence of a double stranded control polynucleotide also having a length of 139 base pairs. Electrophoresis data E2, E4, E6, E8, E9, and E12 were obtained from the TGCE of duplexes prepared by melting an annealing the double stranded sample polynucleotide having a length of 139 base pairs in the presence of a double stranded control polynucleotide having a length of 141 base pairs. As can be seen, E2, E4, E6, E8, E9, and E12 each include 4 peaks indicative of the presence of the duplexes. Electrophoresis data E1, E3, E5, E7, E10, and E11 each include only 1 peak. Thus, the sample polynucleotide may be determined to have the same size as the control used to obtain electrophoresis data E1, E3, E5, E7, E10, and E11.

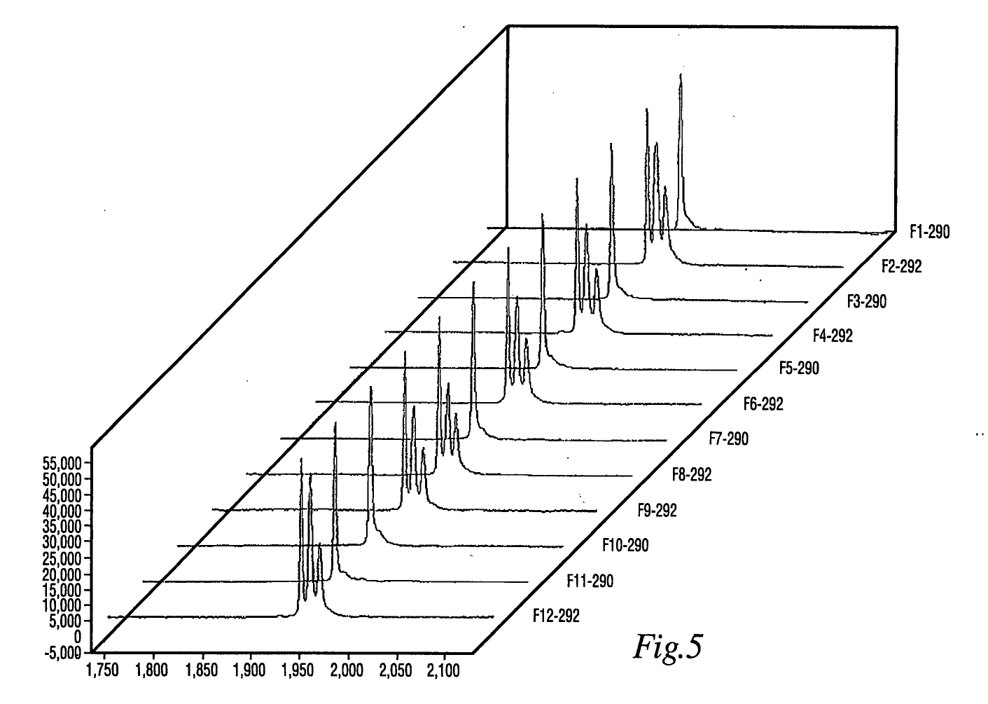

Referring to FIG. 5, 12 sets of simultaneously obtained electrophoresis data, F1-F12 are shown. Electrophoresis data F1, F3, F5, F7, F10, and F11 were obtained from the TGCE of duplexes prepared by melting an annealing a double stranded sample polynucleotide having a length of 290 base pairs in the presence of a double stranded control polynucleotide also having a length of 290 base pairs. Electrophoresis data F2, F4, F6, F8, F9, and F12 were obtained from the TGCE of duplexes prepared by melting an annealing the double stranded sample polynucleotide having a length of 290 base pairs in the presence of a double stranded control polynucleotide having a length of 292 base pairs. As can be seen, F2, F4, F6, F8, F9, and F12 each include 3 peaks indicative of the presence of the duplexes. Electrophoresis data F1, F3, F5, F7, F10, and F11 each include only 1 peak. Thus, the sample polynucleotide may be determined to have the same size as the control used to obtain electrophoresis data F1, F3, F5, F7, F10, and F11. The size determination may also be determined based upon the lower migration rate, and higher migration time of the peaks of electrophoresis data F2, F4, F6, F8, F9, and F12 as compared to electrophoresis data F1, F3, F5, F7, F10, and F11.

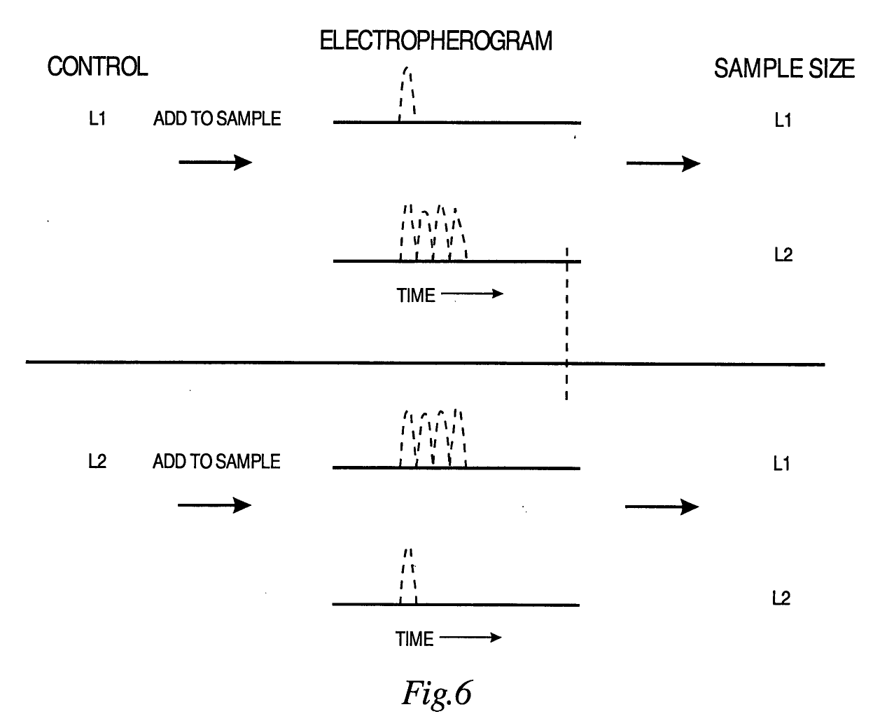

Referring to FIG. 6, an embodiment of a method for determining whether a length of a first polynucleotide is equal to (a) a length of a second polynucleotide or (b) a length of a third polynucleotide is shown. The method comprises, providing a sample comprising a first polynucleotide, which may be, for example, a PCR product. The first polynucleotide is preferably double stranded. A first portion of the of the sample and a control polynucleotide having a length L1 are combined to prepare a first mixture. The control polynucleotide is preferably double stranded. A second portion of the sample and a second control polynucleotide having a length L2 are combined to prepare a second mixture. The second control polynucleotide is preferably double stranded. The control polynucleotide comprises at least 1 additional base than the second control polynucleotide. The at least 1 additional base is located intermediate terminal ends of the control polynucleotide.

First duplexes comprising the first polynucleotide and the control polynucleotide are prepared. Second duplexes comprising the first polynucleotide and the second control polynucleotide are prepared. The first and second duplexes are subjected to temperature gradient electrophoresis to obtain electrophoresis data. The electrophoresis data is analyzed to determine whether the length of the first polynucleotide is equal to (a) the length of the second polynucleotide or (b) the length of the third polynucleotide.

The electrophoresis data may comprise a number N1 first peaks indicative of the presence of the first duplexes, N1 being 1 or greater, and a number N2 second peaks indicative of the presence of the second duplexes, N2 being 1 or greater. The step of analyzing the electrophoresis data may comprise determining N1 and N2. If N1>N2, the length of the first polynucleotide is preferably determined to be equal to the length of the third polynucleotide. If N1<N2, the length of the first polynucleotide is preferably determined to be equal to the length of the second polynucleotide.

The step of analyzing the electrophoresis data may comprise determining a total width of the N1 first peaks and a total width of the N2 second peaks. The total width may be determined, for example, on the basis of a portion of the maximum intensity of the peaks. If the total width of the N1 first peaks > the total width of the N2 second peaks, the length of the first polynucleotide is preferably determined to be equal to the length of the third polynucleotide. If the total width of the N1 first peaks < the total width of the N2 second peaks, the length of the first polynucleotide is preferably determined to be equal to the length of the second polynucleotide.

The step of analyzing the electrophoresis data may comprise determining a migration rate of the N1 first peaks and a migration rate of the N2 second peaks. If the migration rate of the N1 first peaks is > the migration rate of the N2 second peaks, the length of the first polynucleotide is preferably determined to be equal to the length of the third polynucleotide. If the migration rate of the N1 first peaks is < the migration rate of the N2 second peaks, the length of the first polynucleotide is preferably determined to be equal to the length of the second polynucleotide.

The step of analyzing the electrophoresis data may comprise determining a migration time of the N1 first peaks and a migration time of the N2 second peaks. If the migration time of the N1 first peaks is < the migration time of the N2 second peaks, the length of the first polynucleotide is preferably determined to be equal to the length of the third polynucleotide. If the migration time of the N1 first peaks is > the migration time of the N2 second peaks, the length of the first polynucleotide is preferably determined to be equal to the length of the second polynucleotide.

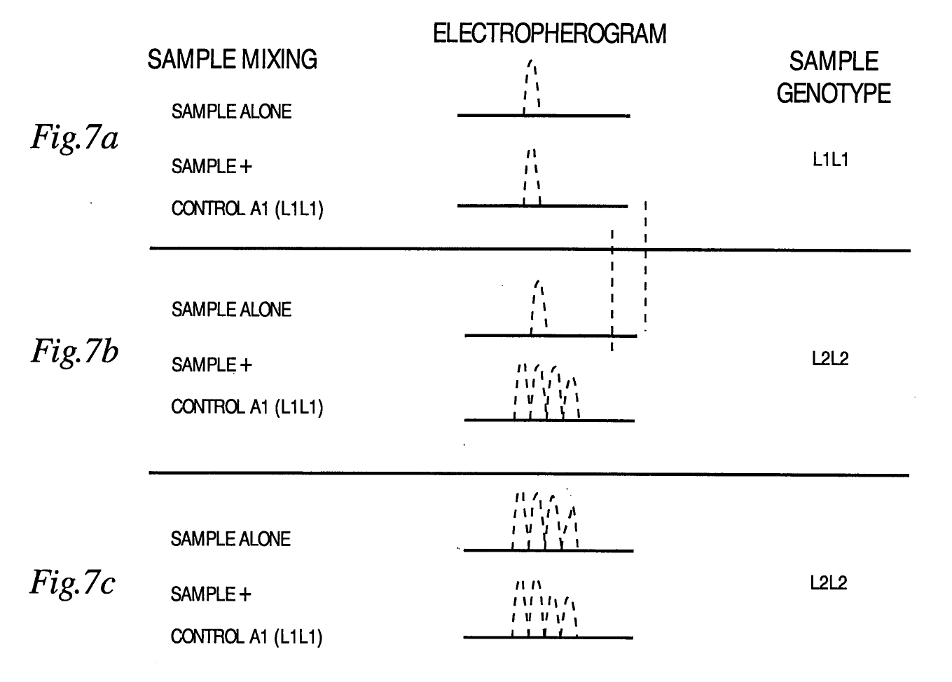

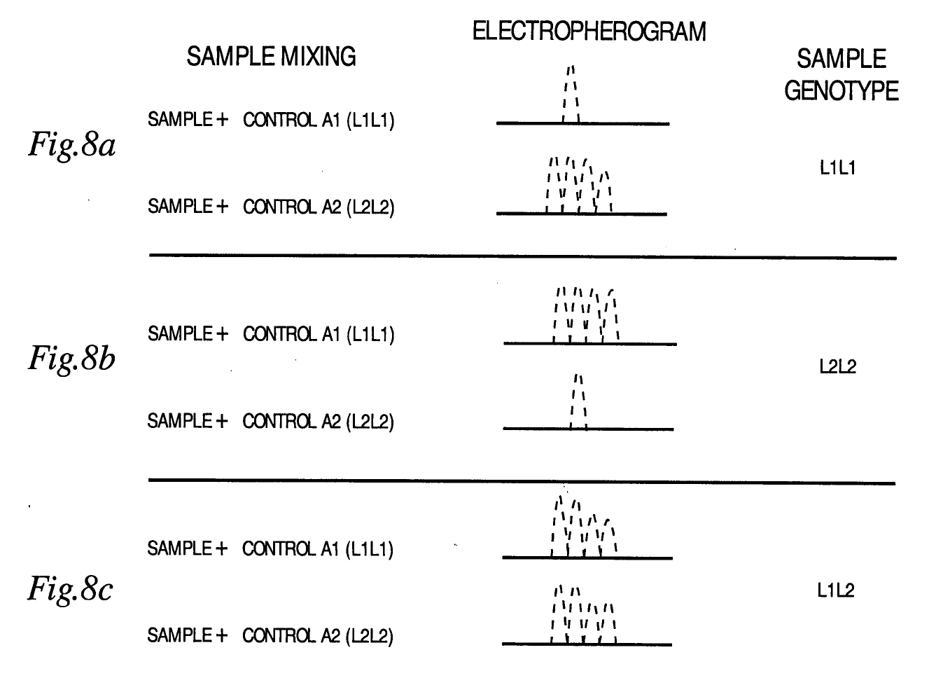

Referring to FIGS. 7 and 8, the assignment of a genotype with a nucleotide length LL to a sample in genetic analysis is shown. In a sample mixing step, the sample polynucleotide is subjected to melting and annealing. In a second sample mixing step, a different portion of sample is subjected to melting and annealing in the presence of a control parent polynucleotide A1 (or sample A1) with a nucleotide length L1 (genotype L1L1). In a third sample mixing step, a different portion of sample is subjected to melting and annealing in the presence of a parent polynucleotide A2 (or sample A2) with a length of L2 (genotype L2L2).

To score all three possible genotypes (i.e. L1L1, L1L2 and L2L2) in a diploid organism, two TGE assays for any testing material may be used to reveal the known DNA variants. Thus, each assay will generate an electrophoresis data for the testing sample. Electrophoresis data obtained from both assays can be combined and produce a final call of genotype for the sample. There are preferred two strategies of performing these two assays. One analyzes the original DNA samples with TGE to obtain a first electropherogram and only adds one of the two homozygous controls to the testing sample to obtain the second electropherogram (FIGS. 7a-7c). The other tests the sample adding each homozygous control to every sample separately (FIGS. 8a-8b).

While the above invention has been described with reference to certain preferred embodiments, it should be kept in mind that the scope of the present invention is not limited to these. Thus, one skilled in the art may find variations of these preferred embodiments which, nevertheless, fall within the spirit of the present invention, whose scope is defined by the claims set forth below.

What is claimed is:

1. A method of determining whether a length of a first polynucleotide is equal to (a) a length of a second polynucleotide or (b) a length of a third polynucleotide, the method comprising:
providing a sample comprising the first polynucleotide;
combining a first portion of the sample with an amount of the second polynucleotide to form a first mixture;
combining a second portion of the sample with an amount of the third polynucleotide to form a second, different mixture, the second polynucleotide comprising at least 1 additional base than the third polynucleotide, the at least 1 additional base being located intermediate terminal ends of the second polynucleotide;
preparing first duplexes comprising the first polynucleotide and the second polynucleotide;
preparing second duplexes comprising the first polynucleotide and the third polynucleotide;
subjecting the first and second duplexes to temperature gradient electrophoresis to obtain electrophoresis data; and
determining whether the length of the first polynucleotide is equal to (a) the length of the second polynucleotide or (b) the length of the third polynucleotide based upon the electrophoresis data.

2. The method of claim 1, wherein the temperature gradient electrophoresis is temperature gradient capillary electrophoresis.

3. The method of claim 2, wherein the second and third polynucleotides each comprise more than 500 bases.

4. The method of claim 3, wherein the second and third polynucleotides each comprise more than 1000 bases.

5. The method of claim 1, wherein the second polynucleotide comprises at least 2 additional bases than the third polynucleotide, the at least 2 additional bases being located intermediate terminal ends of the second polynucleotide.

6. The method of claim 5, wherein the at least 2 additional bases are consecutive.

7. The method of claim 6, wherein the second polynucleotide comprises, intermediate terminal ends of the second polynucleotide, less than 6 additional bases than the third polynucleotide.

8. The method of claim 2, wherein the step of subjecting comprises contacting the first and second duplexes with an intercalating dye during temperature gradient electrophoresis.

9. The method of claim 2, wherein:
the electrophoresis data comprises:
a number N₁ first peaks indicative of the presence of the first duplexes, N₁ being 1 or greater; and
a number N₂ second peaks indicative of the presence of the second duplexes, N₂ being 1 or greater; and
the step of analyzing the electrophoresis data comprises determining N₁ and N₂, wherein (a) if N₁>N₂, the length of the first polynucleotide is determined to be equal to the length of the third polynucleotide and (b) if N₁<N₂, the length of the first polynucleotide is determined to be equal to the length of the second polynucleotide.

10. The method of claim 2, wherein:
the electrophoresis data comprises:
a number N₁ first peaks indicative of the presence of the first duplexes, N₁ being 1 or greater; and
a number N₂ second peaks indicative of the presence of the second duplexes, N₂ being 1 or greater; and
the step of analyzing the electrophoresis data comprises determining a total width of the N₁ first peaks and a total width of the N₂ second peaks, wherein (a) if the total width of the N₁ first peaks > the total width of the N₂ second peaks, the length of the first polynucleotide is determined to be equal to the length of the third polynucleotide and (b) if the total width of the N1 first peaks < the total width of the N₂ second peaks, the length of the first polynucleotide is determined to be equal to the length of the second polynucleotide.

11. The method of claim 2, wherein:
the electrophoresis data comprises:
a number N₁ first peaks indicative of the presence of the first duplexes, N₁ being 1 or greater; and
a number N₂ second peaks indicative of the presence of the second duplexes, N₂ being 1 or greater; and
the step of analyzing the electrophoresis data comprises determining a migration rate of the N₁ first peaks and a migration rate of the N₂ second peaks, wherein (a) if the migration rate of the N₁ first peaks is > the migration rate of the N₂ second peaks, the length of the first polynucleotide is determined to be equal to the length of the third polynucleotide and (b) if the migration rate of the N₁ first peaks is < the migration rate of the N₂ second peaks, the length of the first polynucleotide is determined to be equal to the length of the second polynucleotide.

12. The method of claim 2, wherein:
the electrophoresis data comprises:
a number N₁ first peaks indicative of the presence of the first duplexes, N₁ being 1 or greater; and
a number N₂ second peaks indicative of the presence of the second duplexes, N₂ being 1 or greater; and
the step of analyzing the electrophoresis data comprises determining a migration time of the N₁ first peaks and a migration time of the N₂ second peaks, wherein (a) if the migration time of the N₁ first peaks is < the migration time of the N₂ second peaks, the length of the first polynucleotide is determined to be equal to the length of the third polynucleotide and (b) if the migration time of the N₁ first peaks is > the migration time of the N₂ second peaks, the length of the first polynucleotide is determined to be equal to the length of the second polynucleotide.

13. The method of claim 2, wherein the first and second duplexes are subjected to temperature gradient electrophoresis in different capillaries.

14. The method of claim 2, wherein the first, second, and third polynucleotides are of substantially the same allele.

15. A method of determining a size of a first polynucleotide, comprising:
subjecting a plurality of first duplexes to temperature gradient electrophoresis to obtain first electrophoresis data comprising a number N₁ first peaks indicative of the presence of the first duplexes, N₁ being 1 or greater, wherein each of the first duplexes comprise the first polynucleotide and a second polynucleotide;
subjecting a plurality of second duplexes to temperature gradient electrophoresis to obtain second electrophoresis data comprising a number N₂ second peaks indicative of the presence of the second duplexes, N₂ being 1 or greater, wherein the second duplexes comprise the first polynucleotide and a third polynucleotide, the second polynucleotide comprising at least 1 additional base than the third polynucleotide, the at least 1 additional base being disposed intermediate terminal ends of the second polynucleotide; and
comparing the N₁ first peaks and the N₂ second peaks to determine whether a length of the first polynucleotide is equal to the length of the second polynucleotide or to the length of the third polynucleotide.

16. The method of claim 115, wherein the step of comparing comprises determining N₁ and N₂, wherein (a) if N₁>N₂, the length of the first polynucleotide is determined to be equal to the length of the third polynucleotide and (b) if N₁<N₂, the length of the first polynucleotide is determined to be equal to the length of the second polynucleotide.

17. The method of claim 15, wherein the step of comparing comprises determining a total width of the N₁ first peaks and a total width of the N₂ second peaks, wherein (a) if the total width of the N₁ first peaks > the total width of the N₂ second peaks, the length of the first polynucleotide is determined to be equal to the length of the third polynucleotide and (b) if the total width of the N₁ first peaks < the total width of the N₂ second peaks, the length of the first polynucleotide is determined to be equal to the length of the second polynucleotide.

18. The method of claim 15, wherein the step of comparing comprises determining a migration rate of the N₁ first peaks and a migration rate of the N₂ second peaks, wherein (a) if the migration rate of the N₁ first peaks is < the migration rate of the N₂ second peaks, the length of the first polynucleotide is determined to be equal to the length of the third polynucleotide and (b) if the migration rate of the N first peaks is > the migration rate of the N₂ second peaks, the length of the first polynucleotide is determined to be equal to the length of the second polynucleotide.

19. The method of claim 16, wherein the step of comparing comprises determining a migration time of the N₁ first peaks and a migration time of the N₂ second peaks, wherein (a) if the migration time of the N₁ first peaks is > the migration time of the N₂ second peaks, the length of the first polynucleotide is determined to be equal to the length of the third polynucleotide and (b) if the migration time of the N₁ first peaks is < the migration time of the N₂ second peaks, the length of the first polynucleotide is determined to be equal to the length of the second polynucleotide.

20. The method of claim 15, wherein the steps of subjecting comprise contacting the first and second duplexes with an intercalating dye during temperature gradient electrophoresis.

21. The method of claim 15, wherein the second and third polynucleotides each comprise more than 500 bases.

21. The method of claim 15, wherein the second and third polynucleotides each comprise more than 1000 bases.

22. The method of claim 15, wherein the second polynucleotide comprises at least 2 additional bases than the third polynucleotide, the at least 2 additional bases being located intermediate terminal ends of the second polynucleotide.

23. The method of claim 22, wherein the at least 2 additional bases are consecutive.

24. The method of claim 22, wherein the second polynucleotide comprises, intermediate terminal ends of the second polynucleotide, less than 6 additional bases than the third polynucleotide.

25. The method of claim 15, wherein the first, second, and third polynucleotides are of substantially the same allele.

26. A computer-readable medium comprising executable software code, the code for determining whether a length of a first polynucleotide is equal to (a) a length of a second polynucleotide or (b) a length of a third polynucleotide, the computer readable medium comprising:
code to receive first electrophoresis data, the first electrophoresis data having been obtained by subjecting first duplexes to temperature gradient electrophoresis, the first duplexes comprising the first polynucleotide and, at least partially paired therewith, the second polynucleotide;
code to receive second electrophoresis data, the second electrophoresis data having been obtained by subjecting second duplexes to temperature gradient electrophoresis, the second duplexes comprising the first polynucleotide and, at least partially paired therewith, the third polynucleotide, the second polynucleotide comprising at least 1 additional base than the third polynucleotide, the at least 1 additional base being located intermediate terminal ends of the second polynucleotide;
code to determine the presence of a number N₁ first peaks in the first electrophoresis data, the N₁ first peaks being indicative of the presence of the first duplexes, N₁ being 1 or greater;
code to determine the presence of a number N₂ second peaks in the second electrophoresis data, the N₂ second peaks being indicative of the presence of the second duplexes, N₂ being 1 or greater;
code to determine whether the length of the first polynucleotide is equal to (a) the length of the second polynucleotide or (b) the length of the third polynucleotide based upon the N₁ first peaks and the N₂ second peaks.

27. The computer readable medium of claim 25, wherein the code to determine whether the length of the first polynucleotide is equal to (a) the length of the second polynucleotide or (b) the length of the third polynucleotide comprises:
code to determine the number N1 and the number N2;
code to determine that the length of the first polynucleotide is equal to the length of the third polynucleotide if N₁>N₂; and
code to determine that the length of the first polynucleotide is equal to the length of the second polynucleotide if N₁<N₂.

28. The computer readable medium of claim 25, wherein the code to determine whether the length of the first polynucleotide is equal to (a) the length of the second polynucleotide or (b) the length of the third polynucleotide comprises:
code to determine a total width of the N1 first peaks and a total width of the N₂ second peaks;
code to determine that the length of the first polynucleotide is equal to the length of the third polynucleotide if the total width of the N₁ first peaks > the total width of the N₂ second peaks; and
code to determine that the length of the first polynucleotide is equal to the length of the second polynucleotide if the total width of the N₁ first peaks < the total width of the N₂ second peaks.

29. The computer readable medium of claim 25, wherein the code to determine whether the length of the first polynucleotide is equal to (a) the length of the second polynucleotide or (b) the length of the third polynucleotide comprises:
code to determine a migration rate of the N₁ first peaks and a migration rate of the N₂ second peaks;
code to determine that the length of the first polynucleotide is equal to the length of the second polynucleotide if the migration rate of the N₁ first peaks > the migration rate of the N₂ second peaks; and
code to determine that the length of the first polynucleotide is equal to the length of the third polynucleotide if the migration rate of the N₁ first peaks < the migration rate of the N₂ second peaks.

30. The computer readable medium of claim 25, wherein the code to determine whether the length of the first polynucleotide is equal to (a) the length of the second polynucleotide or (b) the length of the third polynucleotide comprises:
code to determine a migration velocity of the N₁ first peaks and a migration velocity of the N₂ second peaks;
code to determine that the length of the first polynucleotide is equal to the length of the second polynucleotide if the migration velocity of the N₁ first peaks < the migration velocity of the N₂ second peaks; and
code to determine that the length of the first polynucleotide is equal to the length of the third polynucleotide if the migration velocity of the N1 first peaks > the migration velocity of the N₂ second peaks.

31. A method of determining a size of a first polynucleotide, comprising:
receiving first electrophoresis data, the first electrophoresis data having been obtained by subjecting a plurality of first duplexes to temperature gradient electrophoresis, the first electrophoresis data comprising a number N₁ first peaks indicative of the presence of the first duplexes, N₁ being 1 or greater, wherein each of the first duplexes comprise the first polynucleotide and a second polynucleotide;
receiving second electrophoresis data, the second electrophoresis data having been obtained by subjecting a plurality of second duplexes to temperature gradient electrophoresis, the second electrophoresis data comprising a number N₂ second peaks indicative of the presence of the second duplexes, N₂ being 1 or greater, wherein the second duplexes comprise the first polynucleotide and a third polynucleotide, the second polynucleotide comprising at least 1 additional base than the third polynucleotide, the at least 1 additional base being disposed intermediate terminal ends of the second polynucleotide; and
comparing the N₁ first peaks and the N₂ second peaks to determine whether a length of the first polynucleotide is equal to the length of the second polynucleotide or to the length of the third polynucleotide.

32. A method for determining whether a sample of DNA is of a first or second genotype, the DNA comprising a first polynucleotide having a length L₁ and a second polynucleotide having a length L₂, wherein (a) if the DNA is of the first genotype, L₁ and L₂ are the same and the first and second polynucleotides are sufficiently complementary to form a first duplex and (b) if the DNA is of the second genotype, the second polynucleotide comprises at least 1 additional base than the first polynucleotide, the at least 1 additional base being located intermediate terminal ends of the second polynucleotide so that a second duplex comprising the first and second polynucleotides comprises an unpaired region associated with the at least 1 additional base, the unpaired region being located intermediate terminal ends of the second duplex, the method comprising:
combining the first and second polynucleotides with a first control sample comprising a third polynucleotide and a complementary fourth polynucleotide to prepare a first mixture, both the third and the fourth polynucleotides having the same length, either L₁ or L₂, so that a duplex comprising the third and fourth polynucleotides lacks an unpaired region located intermediate terminal ends of the duplex;
subjecting combined polynucleotides of the first mixture to at least one melting step and one annealing step to prepare a second mixture comprising (a) a duplex comprising the first polynucleotide and one of the third and fourth polynucleotides and (b) a duplex comprising the second polynucleotide and the other of the third and fourth polynucleotides;
subjecting the duplexes of the second mixture to temperature gradient electrophoresis to obtain first electrophoresis data; and
determining whether the DNA is of the first or second genotype based on the first electrophoresis data.

33. The method of claim 32, further comprising:
combining the first and second polynucleotides with a second control sample comprising a fifth polynucleotide and a complementary sixth polynucleotide to prepare a third mixture, both the fifth and the sixth polynucleotides have the same length L₁ if the third and fourth polynucleotides have length L₂ or the same length L₂ if the third and fourth polynucleotides have length L1, so that a duplex comprising the fifth and sixth polynucleotides lacks an unpaired region located intermediate terminal ends of the duplex;
subjecting combined polynucleotides of the third mixture to at least one melting step and one annealing step to prepare a fourth mixture comprising (a) a duplex comprising the first polynucleotide and one of the fifth and sixth polynucleotides and (b) a duplex comprising the second polynucleotide and the other of the fifth and sixth polynucleotides;
subjecting the duplexes of the fourth mixture to temperature gradient electrophoresis to obtain second electrophoresis data; and
determining whether the DNA is of the first or second genotype based on both the first and the second electrophoresis data.

33. The method of claim 33, wherein the first electrophoresis data comprises a number N₁ peaks indicative of the presence of the duplexes of the second mixture, N₁ being 1 or greater, the N₁ peaks of the first electrophoresis data having respective first intensities, and the second electrophoresis data comprises a number N₂ peaks indicative of the presence of the duplexes of the fourth mixture, N₂ being 1 or greater, the N₂ peaks of the second electrophoresis data having respective second intensities, and the method comprises:
determining N₁ and N₂;
determining the respective first and second intensitites; and
wherein determining whether the DNA is of the first or second genotype based upon at least 1 of N₁, N₂, the respective first intensities, and the respective second intensities.

33. The method of claim 32, further comprising:
subjecting the DNA, in the absence of the third and fourth polynucleotides, to at least one melting step and at least one annealing step;
subjecting the melted annealed DNA to temperature gradient electrophoresis to obtain sample electrophoresis data; and
determining whether the DNA is of the first or second genotype based on both the first and third electrophoresis data.
